(19) United States
(12) Patent Application Publication
(10) Pub. No.: US 2022/0031216 A1
(43) Pub. Date: Feb. 3, 2022

(54) MEDICAL-MAPPING DEVICE

(71) Applicant: Baylis Medical Company Inc., Montreal (CA)

(72) Inventor: Linus Leung, Toronto (CA)

(21) Appl. No.: 17/386,688

(22) Filed: Jul. 28, 2021

Related U.S. Application Data
(60) Provisional application No. 63/058,811, filed on Jul. 30, 2020.

(51) Int. Cl. A61B 5/287; A61B 5/00; A61B 5/367

(52) U.S. Cl. CPC A61B 5/287; A61B 2562/0209; A61B 5/367; A61B 5/6858

(57) ABSTRACT

A medical-mapping device includes an elongated electrode-support assembly having spaced-apart electrodes configured to be maneuverable, at least in part, into, and along, a confined space of a patient. The spaced-apart electrodes are configured to be selectively movable between a storage position and a deployment position.

### TECHNICAL FIELD

This document relates to the technical field of (and is not limited to) (A) a medical-mapping device (and/or method therefor); and/or (B) a medical-mapping device including an elongated electrode-support assembly having spaced-apart electrodes (and/or method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with the existing (known) medical-mapping device. After much study of, and experimentation with, the existing (known) medical-mapping device, an understanding (at least in part) of the problem and its solution have been identified (at least in part) and are articulated (at least in part) as follows:

Known cardiac mapping is a part of the known radiofrequency ablation procedural flow (procedure). There are known medical-mapping devices configured for assisting in the process of mapping a biological feature (such as the atrium of the heart) of the patient, to thereby facilitate treatment (such as, ablation of heart tissues to treat atrial fibrillation, etc.). Known medical-mapping devices may have a single hoop or multiple prongs. Known medical-imaging systems may rely on these known prongs (etc.) to reach biological features (such as the atrium of the heart); moreover, not all areas, unfortunately, may be reached with the known medical-mapping devices.

It may be desirable to improve, depending on the application, the efficiency of a procedure with an improvement to the known medical-mapping devices.

It may be desirable to provide a medical-mapping device configured to allow for biological features to be more easily reached for viewing by the known medical mapping systems.

To mitigate, at least in part, at least one problem associated with the existing technology, there is provided (in accordance with a major aspect) an apparatus. The apparatus includes and is not limited to (comprises) a medical-mapping device including an elongated electrode-support assembly having spaced-apart electrodes configured to be maneuverable, at least in part, into, and along, a confined space of a patient. The spaced-apart electrodes are configured to be selectively movable between a storage position and a deployment position.

To mitigate, at least in part, at least one problem associated with the existing technology, there is provided (in accordance with a major aspect) a method. The method is for using a medical-mapping device. The method includes and is not limited to (comprises) maneuvering, at least in part, the medical-mapping device having an elongated electrode-support assembly and spaced-apart electrodes into, and along, a confined space of a patient, in which the spaced-apart electrodes are mounted to the elongated electrode-support assembly. The method also includes selectively moving the spaced-apart electrodes between a storage position and a deployment position after the elongated electrode-support assembly and the spaced-apart electrodes are maneuvered, at least in part, into, and along, the confined space of the patient.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 and FIG. 2 depict side views of embodiments (implementations) of a medical-mapping device; and

FIG. 3 and FIG. 4 depict cross-sectional views of embodiments (implementations) of the medical-mapping device of FIG. 1 and FIG. 2 (respectively); and

FIG. 5 and FIG. 6 depict side views of embodiments (implementations) of the medical-mapping device of FIG. 1; and

FIG. 7 and FIG. 8 depict side views of embodiments (implementations) of the medical-mapping device of FIG. 1; and

FIG. 9 depicts a side view of an embodiment (implementation) of the medical-mapping device of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

medical-mapping device 100
electrode-support assembly 102
spaced-apart electrodes (104A, 104B)
electrode-moving assembly 108
catheter assembly 200
distal section 202
catheter lumen 204
inflation lumen 206
first catheter 211
second catheter 212
first distal section221
second distal section 222
first catheter lumen 231
second catheter lumen 232
balloon assembly 300
mesh assembly 400
wire 404
connection 406
auxiliary device 902

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

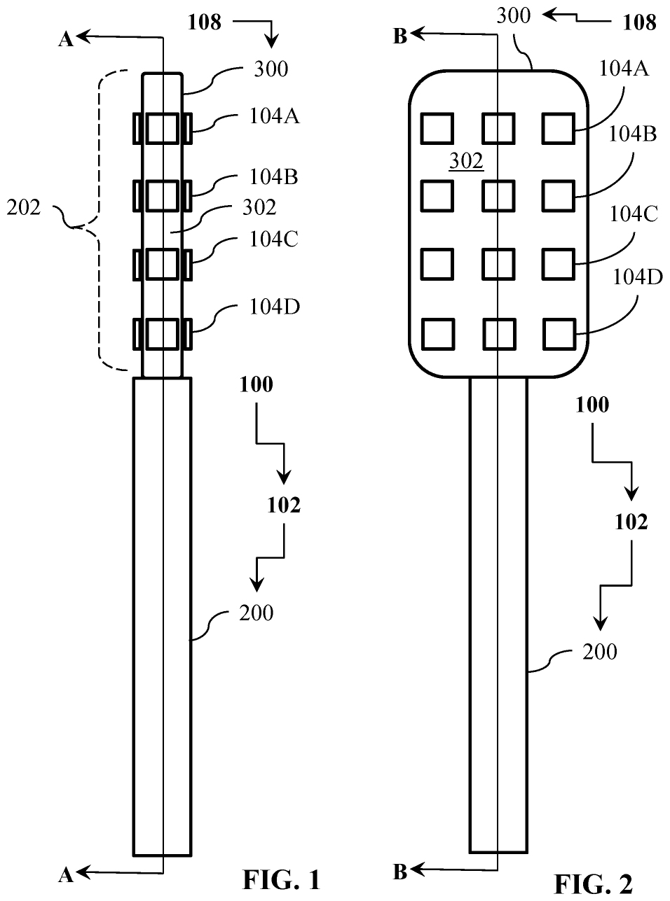

FIG. 1 and FIG. 2 depict side views of embodiments (implementations) of a medical-mapping device 100.

Referring to the embodiments (implementations) as depicted in FIG. 1 and FIG. 2 (and also depicted in the remaining FIGS.), the medical-mapping device 100 may be utilized for electronic anatomical mapping and/or pulmonary vein isolation, etc. The medical-mapping device 100 includes an elongated electrode-support assembly 102 having the spaced-apart electrodes (104A, 104B, 104C, 104D, etc.) configured to be maneuverable, at least in part, into, and along, a confined space (interior space) of a patient. The spaced-apart electrodes (104A, 104B, etc.) are configured to be selectively movable between a storage position (as depicted in FIG. 1) and a deployment position (as depicted in FIG. 2); it will be appreciated that this is done, preferably, after the elongated electrode-support assembly 102 and the spaced-apart electrodes (104A, 104B, etc.) are maneuvered, at least in part, into, and along, the confined space of the patient. There is provided a method for using the spaced-apart electrodes (104A, 104B, etc.). The method includes maneuvering, at least in part, the elongated electrode-support assembly 102 and spaced-apart electrodes (104A, 104B, etc.) into, and along, the confined space of a patient (the spaced-apart electrodes (104A, 104B, etc.) are mounted to the elongated electrode-support assembly 102). The method also includes selectively moving the spaced-apart electrodes (104A, 104B, etc.) between the storage position (as depicted in FIG. 1) and the deployment position (as depicted in FIG. 2); this is done, preferably, after the elongated electrode-support assembly 102 and the spaced-apart electrodes (104A, 104B, etc.) are maneuvered, at least in part, into, and along, the confined space of the patient. It will be appreciated that the spaced-apart electrodes (104A, 104B, etc.) are supported (either directly or indirectly by other intermediate devices, etc.), at least in part, by the elongated electrode-support assembly 102. The medical-mapping device 100 includes components having biocompatible material properties suitable for performance (such as, dielectric strength, thermal, heat and/or electrical insulation, corrosion, water resistance, heat resistance, etc.), for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014]. The medical-mapping device 100 is generally configured to be inserted into a confined space or a tortuous space defined by the body of the patient. The medical-mapping device 100 includes components impermeable by bodily fluids of the patient. The medical-mapping device 100 may be utilized with a medical mapping system such as an electroanatomical mapping system and/or a fluoroscopy mapping system, a nonfluoroscopy mapping system, etc., and any equivalent thereof. The electroanatomical mapping system may include: (A) the CARTO EP (TRADEMARK) mapping system (manufactured by BIOSENSE WEBSTER based in the USA), (B) the ENSITE PRECISION (TRADEMARK) cardiac mapping system (manufactured by Abbott Laboratories based in the USA), (C) the LOCALISA (TRADEMARK) intracardiac mapping system (manufactured by MEDTRONICS INC., based in the USA), and (D) the RHYTHMIA HDx (TRADEMARK) mapping system (manufactured by Boston Scientific Corp., based in the USA).

Referring to the embodiments (implementations) as depicted in FIG. 1 and FIG. 2 (and also depicted in the remaining FIGS.), the spaced-apart electrodes (104A, 104B, etc.) are also configured to be selectively movable between the storage position that is located proximate to the elongated electrode-support assembly 102 (as depicted in FIG. 1) and the deployment position that is located distally from the elongated electrode-support assembly 102 (as depicted in FIG. 2). The method also includes selectively moving the spaced-apart electrodes (104A, 104B, etc.) between the storage position located proximate to the elongated electrode-support assembly 102 (as depicted in FIG. 1) and the deployment position located distally from the elongated electrode-support assembly 102 (as depicted in FIG. 2). The electrode is configured to be detectable by (sensed by) a medical imaging systems, such as an electroanatomical mapping system, for ablating, and/or for pacing/stimulation, etc.

Referring to the embodiments (implementations) as depicted in FIG. 1 and FIG. 2 (and also depicted in the remaining FIGS.), the spaced-apart electrodes (104A, 104B, etc.) are also configured to be selectively movable, at least in part, radially from (away from) the elongated electrode-support assembly 102. This is done, preferably, when moving the spaced-apart electrodes (104A, 104B, etc.) from the storage position (as depicted in FIG. 1) to the deployment position (as depicted in FIG. 2). The spaced-apart electrodes (104A, 104B, etc.) are also configured to be selectively movable, at least in part, radially toward the elongated electrode-support assembly 102; this is done, preferably when moving the spaced-apart electrodes (104A, 104B, etc.) from the deployment position (as depicted in FIG. 2) to the storage position (as depicted in FIG. 1). The method further includes (A) selectively moving, at least in part, the spaced-apart electrodes (104A, 104B, etc.) radially from the elongated electrode-support assembly 102; and (B) selectively moving, at least in part, the spaced-apart electrodes (104A, 104B, etc.) radially toward the elongated electrode-support assembly 102.

Referring to the embodiments (implementations) as depicted in FIG. 1 and FIG. 2 (and also depicted in the remaining FIGS.), the elongated electrode-support assembly 102 includes (in accordance with a preferred embodiment) an elongated catheter assembly 200. The elongated catheter assembly 200 has a distal section 202 configured to be maneuverable, at least in part, into, and along, the confined space of the patient. The distal section 202, of the elongated catheter assembly 200, supports, at least in part, the spaced-apart electrodes (104A, 104B, etc.). The method further includes selectively maneuvering, at least in part, the elongated electrode-support assembly 102 including the elongated catheter assembly 200 having the distal section 202 into, and along, the confined space of the patient.

Referring to the embodiments (implementations) as depicted in FIG. 1 and FIG. 2 (and also depicted in the remaining FIGS.), the elongated electrode-support assembly 102 includes (in accordance with a preferred embodiment) a electrode-moving assembly 108. The electrode-moving assembly 108 is mounted to the distal section 202, of the elongated catheter assembly 200. The electrode-moving assembly 108 is configured to selectively move the spaced-apart electrodes (104A, 104B, etc.) between the storage position (as depicted in FIG. 1) and the deployment position (as depicted in FIG. 2). The method further includes selectively moving the electrode-moving assembly 108 (that is mounted to the distal section 202 of the elongated catheter assembly 200) to selectively move the spaced-apart electrodes (104A, 104B, etc.) between the storage position and the deployment position.

Referring to the embodiments (implementations) as depicted in FIG. 1 and FIG. 2 (and also depicted in the remaining FIGS.), the electrode-moving assembly 108 is also configured to be expandable for selective movement of the spaced-apart electrodes (104A, 104B, etc.) from the storage position (as depicted in FIG. 1) toward the deployment position (as depicted in FIG. 2). The method also includes expanding the electrode-moving assembly 108 for selective movement of the spaced-apart electrodes (104A, 104B, etc.) from the storage position to the deployment position.

Referring to the embodiments (implementations) as depicted in FIG. 1 and FIG. 2 (and also depicted in the remaining FIGS.), the electrode-moving assembly 108 is also configured to be contractible for selective movement of the spaced-apart electrodes (104A, 104B, etc.) from the deployment position (as depicted in FIG. 2) toward the storage position (as depicted in FIG. 1). The method also includes contracting the electrode-moving assembly 108 for selective movement of the spaced-apart electrodes (104A, 104B, etc.) from the deployment position toward the storage position.

Referring to the embodiments (implementations) as depicted in FIG. 1 and FIG. 2, the electrode-moving assembly 108 includes (and is not limited to) a balloon assembly 300. The balloon assembly 300 is mounted to the distal section 202 of the elongated catheter assembly 200. The spaced-apart electrodes (104A, 104B, etc.) are mounted to an outer surface 302 of the balloon assembly 300. Depending on the sensitivity of the electrodes, the spaced-apart electrodes (104A, 104B, etc.) may also be attached directly to the outer surface of the balloon assembly 300.

Referring to the embodiments (implementations) as depicted in FIG. 1 and FIG. 2, the elongated electrode-support assembly 102 includes an elongated catheter assembly 200. The elongated catheter assembly 200 has a distal section 202. A electrode-moving assembly 108 is mounted to the distal section 202 of the elongated catheter assembly 200. The electrode-moving assembly 108 includes a balloon assembly 300 mounted to the distal section 202 of the elongated catheter assembly 200.

Referring to the embodiments (implementations) as depicted in FIG. 1 and FIG. 2, the spaced-apart electrodes (104A, 104B, etc.) (also called an electrode array) are mounted to (on) the balloon assembly 300. The balloon assembly 300 is positioned at a distal tip portion of the catheter assembly 200 to increase the volume of space covered by the spaced-apart electrodes (104A, 104B, etc.). The effective mapping volume can be spatially adjustable by the balloon assembly 300 to various sizes as may be desired. The spaced-apart electrodes (104A, 104B, etc.) mounted to the balloon assembly 300 may cover a larger volume to increase efficiency of the procedure. The spaced-apart electrodes (104A, 104B, etc.) may be used for dual purposes, for both medical-image mapping and/or ablation (and, in this manner, workflow efficiency may be further improved). The spaced-apart electrodes (104A, 104B, etc.) may include at least one or more electrodes configured for image mapping. The spaced-apart electrodes (104A, 104B, etc.) may include at least one or more electrodes configured for ablation. The spaced-apart electrodes (104A, 104B, etc.) may include at least one or more electrodes configured for image mapping and at least one or more electrodes configured for ablation. For instance, for combining ablation tasks and mapping tasks, the two different functions may be separated into two devices that are compatible with each other so that one can be inserted into the other. It will be appreciated that a steering mechanism for the medical-mapping device 100 may help in controlling the movements (selective movements) of the spaced-apart electrodes (104A, 104B, etc.), as may be required during a procedure, etc. The individual electrodes (electrodes) of the spaced-apart electrodes (104A, 104B, etc.) may include (A) flexible electronics, (B) metallic electrodes (either coated, painted or plated), and/or (C) conductive polymer electrodes, etc., and any equivalent thereof. Different types of the spaced-apart electrodes (104A, 104B, etc.) may be used (such as polymeric, metallic, flexible printed circuit boards, etc., and any equivalent thereof). The electrodes of the spaced-apart electrodes (104A, 104B, etc.) may be electrically isolated to provide the individual signals (if desired). Each individual electrode of the spaced-apart electrodes (104A, 104B, etc.) are (preferably) connected to an insulated (respective) wire to convey electrode signals along a shaft and to the elongated electrode-support assembly 102, etc. It will be appreciated that the spaced-apart electrodes (104A, 104B, etc.) are configured to convey (transmit) electrode signals either wirelessly and/or via physical wires, etc. The density and the size of the spaced-apart electrodes (104A, 104B, etc.) may be changed if so desired. The elongated electrode-support assembly 102 may be configured to house the wires (known and not depicted) running from the spaced-apart electrodes (104A, 104B, etc.), where the wires may need to be crimped and/or attached to a cable with a connector (known and not depicted). For instance, the cable may be configured to be connected to an interface unit of a medical-imaging system, etc. It will be appreciated that in accordance with an alternative embodiment, the shape of the balloon assembly 300 may be varied to optimize the mapping or treatment process. For example, there may be multiple instances of the balloon assembly 300 positioned along the distal portion of the shaft. Flat shaped, oblong shaped, spherical shaped, cylindrical shaped, etc. instances of the balloon assembly 300 may be used, etc., if so desired. It will be appreciated that in accordance with another alternative embodiment, multiple instances of the balloon assembly 300 may be utilized (if so desired).

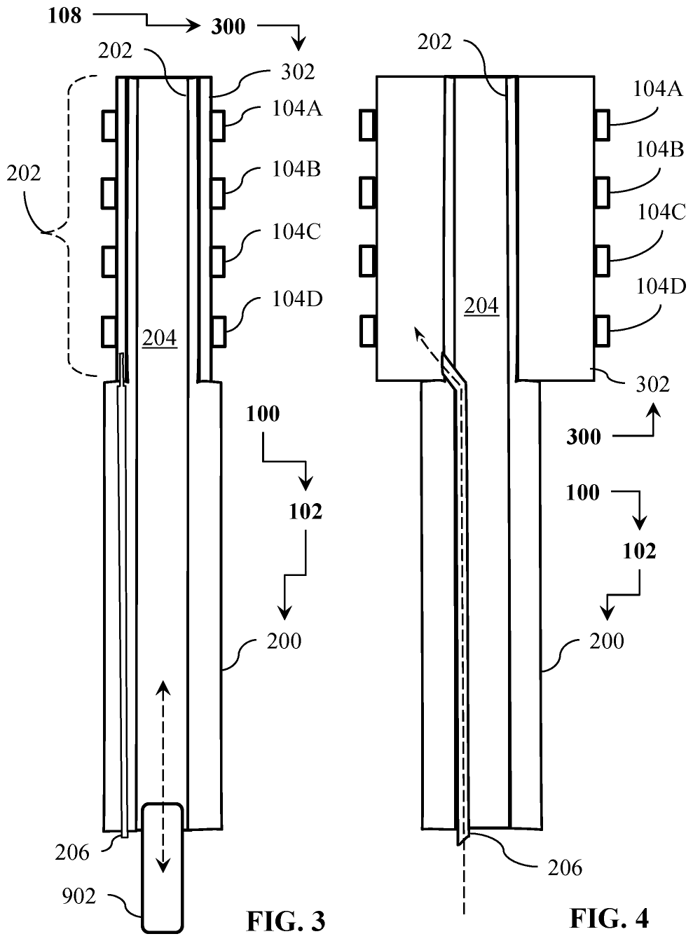

FIG. 3 and FIG. 4 depict cross-sectional views of embodiments (implementations) of the medical-mapping device 100 of FIG. 1 and FIG. 2 (respectively). The cross-sectional views (of FIG. 3 and FIG. 4) are taken along a cross-sectional line A-A of FIG. 1 and a cross-sectional line B-B of FIG. 2 (respectively).

Referring to the embodiment (implementations) as depicted in FIG. 3, the elongated catheter assembly 200 defines an inflation lumen 206. The inflation lumen 206 is configured to be in fluid communication with an interior of the balloon assembly 300. The balloon assembly 300 is configured to be inflated in response to the application of fluid pressure to the inflation lumen 206 (which then pressurizes the interior of the balloon assembly 300). It will be appreciated that, in accordance with an alternative embodiment, the mesh assembly 400 and the balloon assembly 300 are combined, the balloon assembly 300 is configured to be attached to the mesh assembly 400 for deployment; this is done in such a way that when the mesh assembly 400, in use, exits the catheter (similar to the embodiment associated with the mesh assembly 400), the balloon assembly 300 expands (is configured to expand) in response to expansion of the mesh assembly 400 (and, preferably, expansion of the balloon assembly 300 is not a result of fluid injection into the balloon assembly 300).

Referring to the embodiment (implementation) as depicted in FIG. 3, the inflation lumen 206 is received in, at least in part, a length of a catheter lumen 204 defined by the elongated catheter assembly 200. The inflation lumen 206 is configured to be in fluid communication with an interior of the balloon assembly 300. The balloon assembly 300 is configured to be inflated in response to the application of pressure to the interior of the inflation lumen 206. The inflation lumen 206. Is in fluid communication with the interior of the balloon assembly 300.

Referring to the embodiment (implementation) as depicted in FIG. 3, an auxiliary device 902 may be deployed (if desired) along the catheter lumen 204 of the catheter assembly 200, etc.

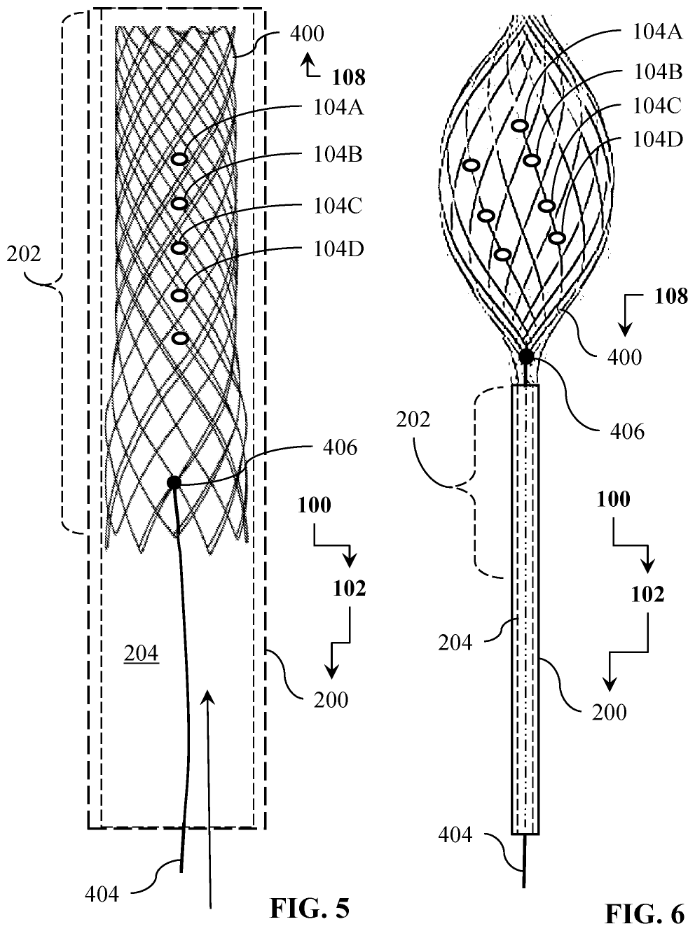

FIG. 5 and FIG. 6 depict side views of embodiments (implementations) of the medical-mapping device 100 of FIG. 1.

Referring to the embodiments (implementations) as depicted in FIG. 5 and FIG. 6, the electrode-moving assembly 108 includes (and is not limited to) a mesh assembly 400. The mesh assembly 400 is mounted to the distal section 202 of the elongated catheter assembly 200. The spaced-apart electrodes (104A, 104B, etc.) are mounted to an outer structure of the mesh assembly 400.

Referring to the embodiments (implementations) as depicted in FIG. 5 and FIG. 6, the elongated electrode-support assembly 102 includes the elongated catheter assembly 200 having the distal section 202. The electrode-moving assembly 108 is mounted to the distal section 202 of the elongated catheter assembly 200. The electrode-moving assembly 108 includes a mesh assembly 400. The mesh assembly 400 is configured to be positioned within a catheter lumen 204 of the elongated catheter assembly 200. The mesh assembly 400 is also configured to be movable from a storage position (that is located within the elongated catheter assembly 200, as depicted in FIG. 5) and a deployment position (that is located outside the elongated catheter assembly 200, as depicted in FIG. 6). The mesh assembly 400 is also configured to be deployed from the distal section 202 of the elongated catheter assembly 200. The spaced-apart electrodes (104A, 104B, etc.) are mounted to an outer structure of the mesh assembly 400.

Referring to the embodiments (implementations) as depicted in FIG. 5 and FIG. 6, a wire 404 (also called a pull wire) is connected to the mesh assembly 400. The wire 404 is configured to urge movement of the mesh assembly 400 between the storage position (as depicted in FIG. 5) and the deployment position (as depicted in FIG. 6) in response to the application of a movement force (such as, a user-initialed movement force) to the wire 404.

Referring to the embodiment (implementation) as depicted in FIG. 5, the wire 404 may be attached to the mesh assembly 400 (such as, attached to a base portion of the mesh assembly 400). The wire 404 may be aligned (at least in part) within, and along and inside, the catheter lumen 204 of the catheter assembly 200. In response to movement of the wire 404, the mesh assembly 400 is urged to move out (from the storage position) from the interior of the catheter lumen 204 of the catheter assembly 200. For this case, the mesh assembly 400 is no longer constrained by the catheter assembly 200 (once deployed as depicted in FIG. 6). The mesh assembly 400 is configured to be compressed into a stressed state (as depicted in FIG. 5). The mesh assembly 400 is configured to expand to an unstressed state (or a natural formation, as depicted in FIG. 6). The mesh assembly 400 is configured to be (preferably) self-deployable.

Referring to the embodiment (implementation) as depicted in FIG. 5, the mesh assembly 400 may include, in accordance with a preferred embodiment, a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus applied to the shape-memory material. This is known as the shape memory effect (SME). Superelasticity (in alloys) may be observed once the shape-memory material is deformed under the presence (an application) of a stimulus force. It will be appreciated that the mesh assembly 400 may form any suitable shape (oval, cylindrical, etc.).

Referring to the embodiments (implementations) as depicted in FIG. 5 and FIG. 6, in response to movement of the wire 404 (such as pulling the wire 404), the mesh assembly 400 may be retracted (moved) into the catheter lumen 204 of the catheter assembly 200, and in response to such movements, the mesh assembly 400 becomes contracted (as depicted in FIG. 5). The mesh assembly 400 is initially contained in the interior of the catheter assembly 200 (as depicted in FIG. 5). The wire 404 is moved (pushed, etc.) out to expose the mesh assembly 400 (as depicted in FIG. 6). The mesh assembly 400 is biased to open to a relaxed state (as depicted in FIG. 6).

Referring to the embodiments (implementations) as depicted in FIG. 5 and FIG. 6, the mesh assembly 400 is configured to be flexible; however, a semi-rigid structure may be necessary for some applications where stiffness may be required to allow for contact between the spaced-apart electrodes (104A, 104B, etc.) and the biological tissue of the patient. The shape of the mesh assembly 400 may be adapted as required for a specific procedure. The mesh assembly 400 may include, for instance, a nitinol mesh (to cover the entirety of the heart chamber). The spaced-apart electrodes (104A, 104B, etc.) are, preferably, distributed (evenly distributed) on the mesh assembly 400, and may potentially assist in the mapping of a larger portion of the heart, etc.

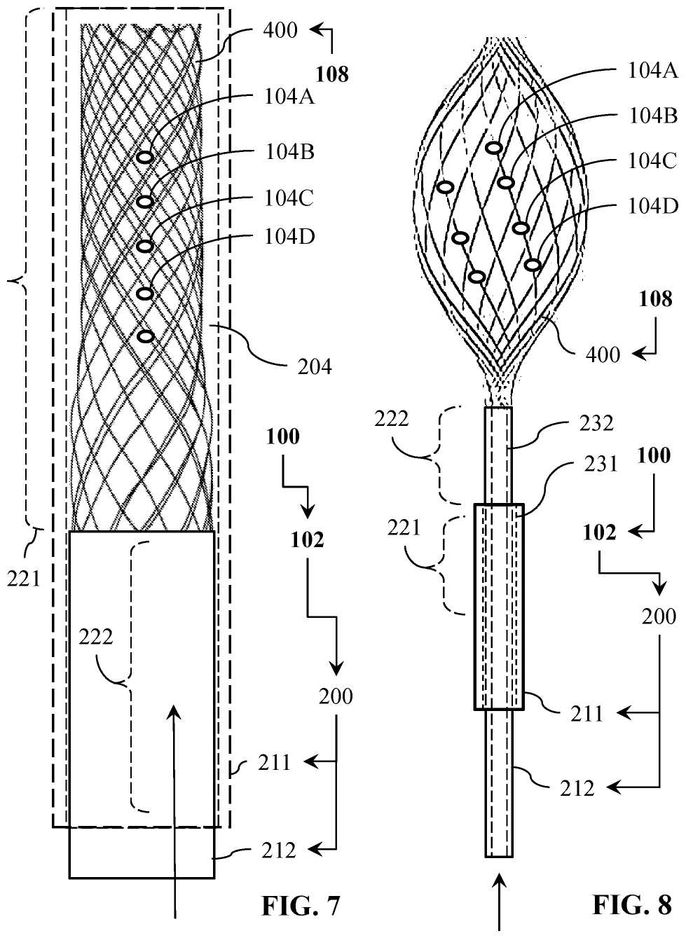

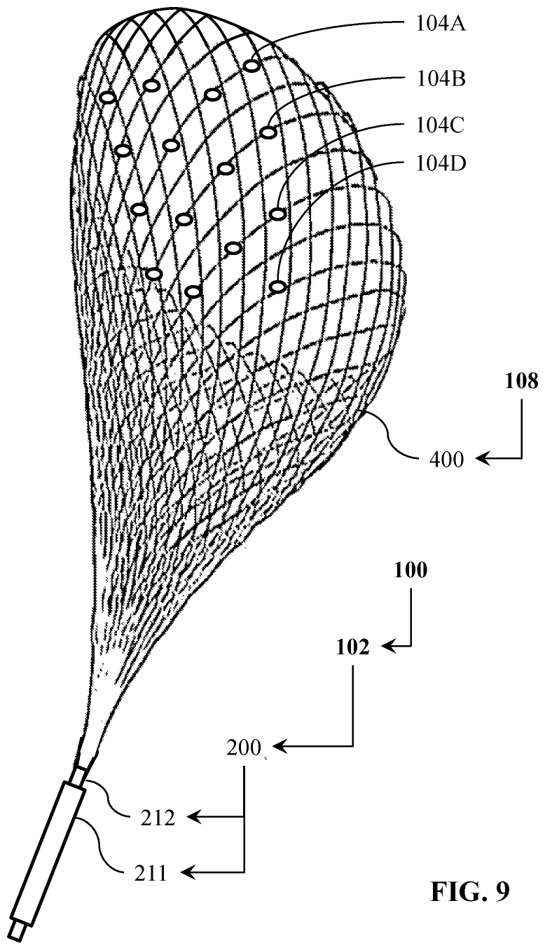

FIG. 7, FIG. 8 and FIG. 9 depict side views of embodiments (implementations) of the medical-mapping device 100 of FIG. 1.

Referring to the embodiments (implementations) as depicted in FIG. 7 and FIG. 8, the elongated electrode-support assembly 102 includes an elongated catheter assembly 200 having a distal section 202. A electrode-moving assembly 108 is mounted to the distal section 202 of the elongated catheter assembly 200. Preferably, the elongated catheter assembly 200 includes a first catheter 211 defining a first catheter lumen 231, and having a first distal section 221. Preferably, the elongated catheter assembly 200 also includes a second catheter 212 having a second distal section 222. The second catheter 212 is receivable within, and movable along, the first catheter lumen 231 of the first catheter 211. The electrode-moving assembly 108 includes a mesh assembly 400. The mesh assembly 400 is configured to be positioned within the first catheter lumen 231 of the first catheter 211 (as depicted in FIG. 7, in the storage position). The mesh assembly 400 is configured to be movable from the storage position (that is located within the first catheter 211, as depicted in FIG. 7) and the deployment position (that is located outside the first catheter 211, as depicted in FIG. 8 or FIG. 9).

Referring to the embodiments (implementations) as depicted in FIG. 7 and FIG. 8, the second catheter 212 is connected to the mesh assembly 400. The second catheter 212 is configured to urge movement of the mesh assembly 400 between the storage position and the deployment position in response to the application of a movement force to the second catheter 212. The second catheter 212 (preferably) defines the second catheter lumen 232 (if required).

Referring to the embodiments (implementations) as depicted in FIG. 7 and FIG. 8, the first catheter 211 and the second catheter 212 are configured to be aligned along a common longitudinal axis. The first catheter 211 and the second catheter 212 are configured to be slidable relative to each other (after the first catheter 211 and the second catheter 212 are aligned along a common longitudinal axis, and the second catheter 212 is received (at least in part) within the first catheter 211). The mesh assembly 400 is initially contained in the first catheter 211 (also called an outer tube) and then the second catheter 212 (also called an inner tube) is moved (pushed out, etc.) from the interior of the first catheter 211 to expose the mesh assembly 400. The mesh assembly 400 is biased to open to a relaxed state (as depicted in FIG. 8 or FIG. 9). The shape of the mesh assembly 400 may be any suitable shape (FIG. 8 and FIG. 9 depict possible shapes for the mesh assembly 400).

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus, comprising:
a medical-mapping device, including:
an elongated electrode-support assembly having spaced-apart electrodes configured to be maneuverable, at least in part, into, and along, a confined space of a patient; and
the spaced-apart electrodes being configured to be selectively movable between a storage position and a deployment position.

2. The apparatus of claim 1, wherein:
the spaced-apart electrodes are also configured to be selectively movable between the storage position located proximate to the elongated electrode-support assembly and the deployment position located distally from the elongated electrode-support assembly.

3. The apparatus of claim 1, wherein:
the spaced-apart electrodes are also configured to be selectively movable, at least in part, radially from the elongated electrode-support assembly; and
the spaced-apart electrodes are also configured to be selectively movable, at least in part, radially toward the elongated electrode-support assembly.

4. The apparatus of claim 1, further comprising:
the elongated electrode-support assembly includes:
an elongated catheter assembly having a distal section configured to be maneuverable, at least in part, into, and along, the confined space of the patient; and
the distal section, of the elongated catheter assembly, supporting, at least in part, the spaced-apart electrodes.

5. The apparatus of claim 4, further comprising:
the elongated electrode-support assembly includes:
a electrode-moving assembly mounted to the distal section, of the elongated catheter assembly; and
the electrode-moving assembly configured to selectively move, at least in part, the spaced-apart electrodes between the storage position and the deployment position.

6. The apparatus of claim 5, wherein:
the electrode-moving assembly is also configured to be expandable for selective movement of the spaced-apart electrodes from the storage position toward the deployment position.

7. The apparatus of claim 5, wherein:
the electrode-moving assembly is also configured to be contractible for selective movement of the spaced-apart electrodes from the deployment position toward the storage position.

8. The apparatus of claim 1, wherein:
the elongated electrode-support assembly includes:
an elongated catheter assembly having a distal section; and
a electrode-moving assembly mounted to the distal section, of the elongated catheter assembly; and
wherein the electrode-moving assembly includes:
a balloon assembly mounted to the distal section of the elongated catheter assembly.

9. The apparatus of claim 8, wherein:
the elongated catheter assembly defines an inflation lumen configured to be in fluid communication with an interior of the balloon assembly; and
the balloon assembly is configured to be inflated in response to application of fluid pressure to the inflation lumen.

10. The apparatus of claim 8, wherein:
an inflation lumen is received in, at least in part, a length of a catheter lumen defined by the elongated catheter assembly; and
the inflation lumen is configured to be in fluid communication with an interior of the balloon assembly; and
the balloon assembly is configured to be inflated in response to application of fluid pressure to the inflation lumen.

11. The apparatus of claim 8, wherein:
the spaced-apart electrodes are mounted to an outer surface of the balloon assembly.

12. The apparatus of claim 1, wherein:
the elongated electrode-support assembly includes:
an elongated catheter assembly having a distal section; and
a electrode-moving assembly mounted to the distal section, of the elongated catheter assembly; and
wherein the electrode-moving assembly includes:
a mesh assembly being configured to be positioned within a catheter lumen of the elongated catheter assembly; and
the mesh assembly configured to be movable from the storage position located within the elongated catheter assembly to the deployment position located outside the elongated catheter assembly.

13. The apparatus of claim 12, further comprising:
a wire connected to the mesh assembly; and
the wire configured to urge movement of the mesh assembly between the storage position and the deployment position in response to application of a movement force to the wire.

14. The apparatus of claim 12, wherein:
the spaced-apart electrodes are mounted to an outer structure of the mesh assembly.

15. The apparatus of claim 1, wherein:
the elongated electrode-support assembly includes:
an elongated catheter assembly having a distal section; and
a electrode-moving assembly mounted to the distal section of the elongated catheter assembly; and
wherein the elongated catheter assembly includes:
a first catheter defining a first catheter lumen, and having a first distal section; and
a second catheter having a second distal section; and
the second catheter receivable within, and movable along, the first catheter lumen of the first catheter; and
wherein the electrode-moving assembly includes:
a mesh assembly configured to be positioned within the first catheter lumen of the first catheter; and
the mesh assembly is configured to be movable between the storage position located within the first catheter and the deployment position located outside the first catheter.

16. The apparatus of claim 15, wherein:
the second catheter is connected to the mesh assembly; and
the second catheter is configured to urge movement of the mesh assembly between the storage position and the deployment position in response to application of a movement force to the second catheter.

17. The apparatus of claim 15, wherein:
the second catheter defines a second catheter lumen.

18. A method of using a medical-mapping device, the method comprising:
maneuvering, at least in part, the medical-mapping device having an elongated electrode-support assembly and spaced-apart electrodes into, and along, a confined space of a patient, in which the spaced-apart electrodes are mounted to the elongated electrode-support assembly; and
selectively moving the spaced-apart electrodes between a storage position and a deployment position after the elongated electrode-support assembly and the spaced-apart electrodes are maneuvered, at least in part, into, and along, the confined space of the patient.

19. The method of claim 18, further comprising:
selectively moving the spaced-apart electrodes between the storage position located proximate to the elongated electrode-support assembly and the deployment position located distally from the elongated electrode-support assembly.

20. The method of claim 18, further comprising:
selectively moving, at least in part, the spaced-apart electrodes radially from the elongated electrode-support assembly; and
selectively moving, at least in part, the spaced-apart electrodes radially toward the elongated electrode-support assembly.
